(19) United States
(12) Patent Application Publication
(10) Pub. No.: US 2023/0038166 A1
(43) Pub. Date: Feb. 9, 2023

(54) METHODS OF DETECTING LIPOARABINOMANNAN AND DIAGNOSING NONTUBERCULOSIS MYCOBACTERIAL INFECTION

(71) Applicants: NATIONAL JEWISH HEALTH, Denver, CO (US); COLORADO STATE UNIVERSITY RESEARCH FOUNDATION, Fort Collins, CO (US)

(72) Inventors: Jerry Allan NICK, Denver, CO (US); Delphi CHATTERJEE, Fort Collins, CO (US)

(21) Appl. No.: 17/778,355

(22) PCT Filed: Dec. 17, 2020
(86) PCT No.: PCT/US2020/065484

§ 371 Date: May 19, 2022

Related U.S. Application Data
(60) Provisional application No. 62/949,341, filed on Dec. 17, 2019.

(51) Int. Cl. G01N 33/569; G01N 33/92

(52) U.S. Cl. CPC G01N 33/5695; G01N 33/92; G01N 2800/122; G01N 2800/382

(57) ABSTRACT

Methods for detecting a mycobacterial infection in a patient are disclosed. These methods include the step of detecting lipoarabinomannan (LAM) and/or derivatives thereof in a biological sample from the patient. Methods for diagnosing disease, including nontuberculous mycobacterial infection (NTM), and kits for the described methods are also provided.

### CROSS-REFERENCE TO RELATED APPLICATIONS

This application claims the benefit of priority under 35 U.S.C. § 119(e) to U.S. Provisional Patent Application No. 62/949,341, filed Dec. 17, 2019. The entire disclosure of U.S. Provisional Patent Application No. 62/949,341 is incorporated herein by reference.

### STATEMENT OF FEDERALLY SPONSORED RESEARCH

This invention was made with government support under grant numbers AI132680 and HL146228 awarded by the National Institutes of Health. The U.S. government has certain rights in the invention.

### TECHNICAL FIELD

This invention relates generally to methods of detecting lipoarabinomannan that include a novel pretreatment step, methods of diagnosing disease including nontuberculosis mycobacterium infection, and kits for practicing the methods.

### BACKGROUND

Nontuberculous mycobacteria (NTM) are environmental organisms found in soil and water worldwide that can cause chronic lung infection, usually in the context of lung disease or immunodeficiency. Cystic fibrosis (CF) has been identified as the disease population at greatest risk for CF airway infection (Martiniano S L, et al. Nontuberculous mycobacteria in cystic fibrosis: updates and the path forward. Pediatr Pulmonol 2017:52). Within the CF population, the vast majority of infections occur as a result of either species within the M. avium complex (MAC) or subspecies of M abscessus (MABC). In the largest longitudinal survey to date, 20% of CF patients who had NTM cultures obtained over a 5-year interval had a positive culture for NTM (Adjemian J, et al. Epidemiology of Pulmonary Nontuberculous Mycobacterial Sputum Positivity in Patients with Cystic Fibrosis in the United States, 2010-2014. Ann Am Thorac Soc 2018; 15:817-26). Among people with CF, detection of NTM in the sputum is of uncertain significance, as often the infection is cleared without treatment, or remains indolent for years (Martiniano S L, et al. Clinical significance of a first positive nontuberculous mycobacteria culture in cystic fibrosis. Ann Am Thorac Soc 2014; 11:36-44). Following a first positive NTM culture, approximately 40% of persons with CF were subsequently determined to meet American Thoracic Society (ATS) criteria for NTM pulmonary disease and were offered treatment.

In all aspects of CF, the lack of sensitive and specific markers of NTM in the airway is a significant barrier to patient care. Currently, culture from the airway is the only method utilized for screening, and the gold standard by which all diagnosis and treatment decisions are made (Griffith D E, et al. Infectious Disease Society of A. An official ATS/IDSA statement: diagnosis, treatment, and prevention of nontuberculous mycobacterial diseases. Am J Respir Crit Care Med 2007; 175:367-416). Limitations of culture from the airway often include slow growth (up to 8 weeks), high cost, low sensitivity due to required decontamination procedures, and difficulty in obtaining samples in children and non-sputum producers. In addition, a given sputum sample may not reflect the often heterogeneous and compartmentalized nature of NTM in the individual patient, and a positive sputum culture of NTM can lag development of clinical symptoms by up to a year (Martiniano S L, et al. Nontuberculous mycobacteria in cystic fibrosis: updates and the path forward. Pediatr Pulmonol 2017:52). Given the improvements to CF therapy over last two decades and with the growing availability of cystic fibrosis transmembrane conductance regulator (CFTR) modulators, fewer pediatric and even adult patients with CF are able to routinely expectorate sputum. Currently, U.S. Cystic Fibrosis Foundation and European Cystic Fibrosis Society Guidelines recommend annual screening for NTM by sputum (Floto R A, et al. US Cystic Fibrosis Foundation and European Cystic Fibrosis Society consensus recommendations for the management of non-tuberculous mycobacteria in individuals with cystic fibrosis: executive summary. Thorax 2016; 71:88-90.) but given these limitations, only 20% of individuals in the CF Patient Registry met this benchmark over a 5-year period, and 21% had no NTM cultures reported over the same interval (Adjemian J, et al. Epidemiology of Pulmonary Nontuberculous Mycobacterial Sputum Positivity in Patients with Cystic Fibrosis in the United States, 2010-2014. Ann Am Thorac Soc 2018; 15:817-26).

Lipoarabinomannan (LAM) is a cell wall lipoglycan found in all mycobacteria species which is released from metabolically active or degrading cells in the circulation and found in the urine of infected patients. LAM has recently gained attention as a biomarker for active tuberculosis (Gupta-Wright et al. 2016. BMC Med 14:53; Lawn et al. 2015. J Acquir Immune Defic Syndr doi:10.1097/QAI.0000000000000672; Amin, et al. 2018. Tuberculosis (Edinb) 111:178-187; Sigal et al. 2018. J Clin Microbiol 56; Broger et al. 2019. PLoS One 14:e0215443; MacLean, et al. 2019. Nat Microbiol 4:748-758). In the setting of tuberculosis, clinical evaluation of LAM by immunological assays are very promising with high specificity and sensitivity. There have been arguments that there was a compromise with specificity due to a possibility of infection with NTM, and it has not been clearly shown if clinical samples of NTM contain detectable amounts of LAM. Previously, urine LAM were reported to have high specificity, but low sensitivity (91-99% and 9-39%, respectively) for pulmonary NTM in the Danish CF population (Qvist et al. 2014. BMC Infect Dis 14:655).

Thus, there remains a need for improved methods of screening CF patients for NTM infection.

### SUMMARY

One embodiment relates to methods and/or uses for diagnosing nontuberculosis mycobacterial (NTM) infection in a patient by detecting LAM or a derivative of LAM in a biological sample from the patient.

In one aspect, the patient has a respiratory disease or condition is selected from the group consisting of cystic fibrosis (CF), asthma, chronic obstructive pulmonary disease (COPD), bronchiectasis, silicosis, sarcoidosis, pneumoconiosis, and interstitial lung disease. In one aspect, the respiratory disease or condition is CF.

In still another aspect, the biological sample is a urine sample.

In another aspect, detection of LAM or a derivative thereof in the biological sample from the patient diagnoses the patient has having NTM infection, and wherein the patient is treated for the NTM infection and/or further tested for infection in a sputum sample from the patient, and/or monitored for clearance of NTM infection. In yet another aspect, non-detection of the LAM or derivative thereof in the biological sample from the patient diagnoses the patient as not having NTM and wherein the patient is not tested again for the presence of NTM for at least 6 months or at least 1 year.

Another embodiment of the invention relates to methods and/or uses for detecting LAM in a biological sample, by detecting a derivative of LAM in the biological sample. In one aspect, the biological sample is a urine sample. In one aspect, the biological sample is obtained from a patient having a respiratory disease or condition selected from the group consisting of CF, asthma, COPD, bronchiectasis, silicosis, sarcoidosis, pneumoconiosis, and interstitial lung disease. In another aspect, the biological sample is obtained from a cystic fibrosis patient.

In one aspect of any of the methods or uses described above or elsewhere herein, in one aspect, the biological sample and/or urine sample is purified by hydrophobic interaction chromatography (HIC) prior to the detection of the derivative of LAM. The HIC may comprise contacting the sample with OCTYL SEPHAROSE® chromatography media.

In another aspect of any of the methods or uses described above or elsewhere herein, the derivative of LAM is be D-arabinose. The D-arabinose in the sample may be formed by contacting the sample with an acid, such as trifluoracetic acid. In one aspect, the D-arabinose is detected by gas-chromatography/mass spectrometry (GC/MS). In one aspect, the GC/MS further comprises using an internal standard, such as D-UL-¹³C₅-arabinose.

In still another aspect of any of the methods or uses described above or elsewhere herein, the derivative of LAM is tuberculostearic acid (TBSA). The TBSA may be formed by contacting the sample with a base, whereby the biological sample can undergo alkaline hydrolysis. In one aspect, the TBSA is detected by gas-chromatography/mass spectrometry (GC/MS). In one aspect, the GC/MS further comprises using an internal standard, such as D₂-palmitic acid.

In another aspect of any of the methods or uses described above or elsewhereherein, the concentration of LAM and/or derivative thereof in the biological sample is determined. In one aspect, the concentration of the LAM and/or derivatives thereof is detected in the biological sample at concentrations between about 0.01 to about 10,000 ng/mL.

Another embodiment of the invention provides kits for detecting LAM or a derivative thereof in a biological sample using the methods of this disclosure. These kits can include any one or more of OCTYL SEPHAROSE® separation media, acids, bases, internal standards, assay buffers, wash buffers, sample diluents, and standard diluents. Specific internal standards that can be included in these kits include D-¹³C₅-UL-arabinose, D₂-palmitic acid, or a combination thereof. In one aspect, these kits may further comprise detection agents for other known mycobacteria and/or detection agents for other known infectious agents common to patients having a respiratory disease or condition. In one aspect, the respiratory disease or condition is selected from the group consisting of CF, asthma, COPD, bronchiectasis, silicosis, sarcoidosis, pneumoconiosis, and interstitial lung disease. In still another aspect, the respiratory disease or condition is CF.

It is to be appreciated that any feature described herein can be claimed in combination with any other feature(s) as described herein, regardless of whether the features come from the same described embodiment.

This Summary is neither intended nor should it be construed as representative of the full extent and scope of the present disclosure. Moreover, references made herein to “the present disclosure,” or aspects thereof, should be understood to mean certain embodiments of the present invention and should not necessarily be construed as limiting all embodiments to a particular description. The present invention is set forth in various levels of detail in this Summary as well as in the attached drawings and the Detailed Description and no limitation as to the scope of the present invention is intended by either the inclusion or non-inclusion of elements, components, etc. in this Summary. Additional aspects of the present invention will
become more readily apparent from the Detailed Description, particularly when taken together with the figures.

### BRIEF DESCRIPTION OF THE DRAWINGS

FIGS. 1A and 1B show the quantity of urine LAM in patients with CF culture positive for MABSC and MAC. FIG. 1A: TBSA LAM (ng/ml) and FIG. 1B: D-ara LAM (ng/ml) detected by GC/MS for subjects whose most recent sputum NTM culture was identified as MAC or MABSC. Mean TBSA and D-ara quantities were greater in subjects whose most recent sputum NTM species was within MABSC (*p=0.007 and **p=0.03 respectively, by one-way ANOVA). Individuals who have cleared their sputum cultures for more than 1 year are depicted with open squares, while subjects who have had a positive NTM culture within a year are depicted with a circle. Box and whiskers plot represent median, 25^{th}-75^{th} quartile, median and range.

FIGS. 2A and 2B are representative GC/MS chromatograms showing the absence and/or presence of urinary LAM related to NTM-negativity and/or positivity. FIG. 2A: D-Arabinose (D-ara) MS/MS method monitoring m/z 420.9-m/z 192.9): Four characteristic peaks of Internal Standard (¹³C₅-D-Arabinose; top panel); Sequentially (top to bottom), 309 and 308 NTM negative, D-Ara negative; BP201 and 303 are D-Arabinose positive, LAM positive, NTM positive. FIG. 2B: Tuberculostearic acid (TBSA) Single ion monitoring (SIM) at m/z 297.3: TBSA standard (C:19; top panel); Sequentially, 309 and 308 NTM negative, TBSA negative; BP201 and 303 are TBSA positive, LAM positive, NTM positive.

FIGS. 3A-3C shows the results of direct ELISA for selection of antibody for NTM LAM analysis in clinical samples. LAM samples were prepared and purified as described in Shi L, et al. (Lipoglycans of Mycobacterium tuberculosis: isolation, purification, and characterization, p. In: Brown TpaAC, editor. Mycobacteria protocols, 465. Barts and The London, London, UK: Humana Press; 2009. p. 23-45) except LepLAM was purified from M. leprae cells isolated from infected armadillo liver and spleen (Torrelles J B, et al. Truncated Structural Variants of Lipoarabinomannan in Mycobacterium leprae and an Ethambutol-resistant Strain of Mycobacterium tuberculosis. J Biol Chem 2004; 279:41227-39.). A194-01 (FIG. 3C) is a human monoclonal Ab derived from a tuberculosis patient against TBLAM and CS35 (FIG. 3A) and CHCS9-08 (FIG. 3B) were raised against M. leprae whole cells. Reactivity and specificity of the mAbs have been described previously (Amin A G, et al. Detection of lipoarabinomannan in urine and serum of HIV-positive and HIV-negative TB suspects using an improved capture-enzyme linked immuno absorbent assay and gas chromatography/mass spectrometry. Tuberculosis (Ed-inb) 2018; 111:178-87; Choudhary A, et al. Characterization of the Antigenic Heterogeneity of Lipoarabinomannan, the Major Surface Glycolipid of Mycobacterium tuberculosis, and Complexity of Antibody Specificities toward this Antigen. J Immunol 2018; 200:3053-66).

FIG. 4 shows the results of urinary LAM detection by Capture ELISA using specific anti-LAM, mouse and human monoclonal antibodies. CS35 IgG3 mouse monoclonal was used as the capture antibody against A194-01 IgG1 human monoclonal as the detection antibody. BSA was used as the ELISA negative control and M avium LAM (ng/ml) spiked in urine (serially diluted two-fold) from a healthy volunteer was used as the LAM standard curve (bars in grey) to obtain the LoD for the ELISA. A total of 32 samples [n=10 NTM negative CF patients, n=22 microbiologically confirmed NTM positive CF patients] were analyzed. ELISA was performed in duplicates and based on the ELISA cutoff (horizontal line), the samples with OD450 nm above the cutoff were ELISA positive for LAM and below the cutoff were designated as ELISA negative for LAM.

FIG. 5 shows urine LAM analysis of subjects with negative NTM sputum cultures. Subjects (n=14) with no evidence for NTM airway infection with >5 cultures, and no history by chart review. Urine LAM assay collected at day 0 correlated completely with previous negative sputum culture for all subjects. Subject BP011 (top row) developed NTM disease 1.8 years following initial urine LAM (darker circles). A urine LAM analysis at that time changed to positive (darker arrow). The subject was initiated on treatment forts. avium, with subsequent conversion to negative sputum cultures (lighter circles).

### DETAILED DESCRIPTION

This disclosure advantageously provides non-invasive methods and/or uses of detecting LAM and derivatives thereof in a biological sample, such as urine, by Gas Chromatography Mass Spectrometry (GC/MS). These methods may be effectively used as part of a two-step screening/diagnosis strategy for patients having respiratory diseases or conditions including but not limited to CF and/or for patients who are susceptible to NTM infection, such as those who are immunocompromised.

Because approximately 80% of the CF population will not culture NTM over a 5-year interval, urine LAM can be a useful, non-invasive test to screen for individuals with low risk of having a positive culture. As demonstrated herein, using a cross-sectional design of well-characterized CF patients, the utility of urine LAM was tested over a range of clinical situations, ranging from fulminant pulmonary disease with both MAC and MABSC, to individuals verified culture negative throughout their lifetime. Simultaneously two LAM based assays were ran, one assay being an antibody-based immunoassay (TB capture ELISA) and the other a chemometric assay, both developed by the inventors for urinary LAM validation in M tuberculosis diagnostics. The latter method involved use of GC/MS and is impartial to the use of antibodies. Urine LAM predicted the absence of previous positive NTM sputum cultures in all subjects tested.

Further, this disclosure advantageously provides methods and/or uses for detecting mycobacterial infections in a patient having a respiratory disease or condition, wherein the respiratory disease or condition is selected from CF, asthma, chronic obstructive pulmonary disease (COPD), bronchiectasis, silicosis, sarcoidosis, pneumoconiosis, and interstitial lung disease. In a preferred aspect, the patient has CF. In another aspect, the patient is susceptible to NTM infection, including but not limited to an immunocompromised patient. These methods include the detection of LAM or derivatives thereof in a biological sample from the patient. In one aspect, the biological sample is selected from the group consisting of urine, sputum, blood, bronchoalveolar lavage (BAL) and exhaled breath. In a preferred aspect, the biological sample is urine. The LAM can be detected and/or quantitated by the production and detection of derivatives of LAM. These derivatives can be detected by GC/MS. These methods may be effectively used as part of a two-step screening/diagnosis strategy for a patient having a respiratory disease or condition and/or for patients who are susceptible to NTM infection. In a preferred aspect, the patient has CF.

NTM species are known in the art and include but are not limited to slowly growing species (defined as colony formation in greater than 7 days) such as Mycobacterium kansasii, Mycobacterium marinum, Mycobacterium ulcerans, Mycobacterium avium, Mycobacterium intracellulare, Mycobacterium malmoense, Mycobacterium haemophilum, Mycobacterium xenopi, Mycobacterium simiae, as well as rapidly growing species (defined as colony formation in less than 7 days) such as Mycobacterium abscessus, Mycobacterium chelonae, and Mycobacterium fortuitum.

The detection of LAM in urine by GC/MS is complex as normal urine can contain both stereoisomeric forms D- and L-arabinose (from nutrients and food), which complicate the detection of D-arabinose (D-Ara) that is specific to LAM. As disclosed herein, the inventors have developed a sample handling protocol whereby LAM is separated from these endogenous neutral polysaccharides by way of hydrophobic interaction chromatography followed by chiral glycosylation (with R-2-octanol) which efficiently distinguishes D- and L-stereoisomers of arabinose. This protocol also serves the purpose of releasing LAM from its protein complex and to distinguish L- from D-arabinose present in other arabinose containing neutral polysaccharide(s). An internal standard is then added (¹³C-labeled D-Ara, 200 ng) to allow efficient quantification of LAM D-Ara. In addition to estimating D-Ara, a detection method for TBSA, which is present on the anchor region of LAM, in urine has also been developed. The derivatization is performed on the LAM fractions eluted off Octyl Sepharose columns with the rationale that the fractions that contain D-Ara must also have TBSA, pointing to the presence of intact LAM.

As disclosed herein, urine LAM analysis by GC/MS is a useful, non-invasive screening tool to identify patients in which sputum cultures for NTM are not needed, especially those patients with CF. The great majority of people with CF will not develop NTM infection, and in the absence of clinical suspicion, a negative urine LAM result could potentially replace sputum cultures as a method of annual screening. In particular, children are at low risk for NTM infection, and are also often unable to expectorate sputum. Going forward, the use of CFTR modulators promises to significantly reduce the proportion of people with CF who are capable of expectorating sputum at baseline health, while at the same time increasing the life expectancy of the population. Identification of non-invasive, culture-independent markers for NTM infection has been identified as a research priority for the CF population, and urine LAM detection could be part of a lifetime screening strategy for the diagnosis and treatment of NTM. For example, CF patients receiving a negative test result in the testing methods of this disclosure could avoid further confirmatory or other testing for an NTM infection for at least 6 months or a year. Alternatively, CF patients receiving a positive test result in the testing methods of this disclosure could be recommended for a confirmatory test or for treatment of a mycobacterial infection.

The methods and/or uses disclosed herein demonstrate the use of LAM or derivatives thereof to serve as a marker for NTM infection in patients having a respiratory disease or condition and/or for patients who are susceptible to NTM infection. These methods are particularly useful to identify CF patients in which sputum cultures for NTM are not needed, thereby saving time and expense and reducing patient discomfort in collecting or attempting to collect sputum samples. Additionally, these methods could potentially replace sputum cultures as a method of annual screening in CF patients, which would be very helpful, for example, in children who are at low risk for NTM infection and are often unable to expectorate sputum. Currently accepted and rigorous diagnostic methods are microbial culturing and a nucleic acid amplification test (NAAT) test, but both are considered to be too costly and complex for routine use in resource-limited settings. Cost and ease-of-use are pivotal in dictating the deployment of a test in regions of the world where it is needed the most. However, the most important diagnostic need for NTM is the identification and validation of one or more antigens, either individually or in a panel, that can be used for the reliable and early diagnosis of the disease. The experiments described herein demonstrate that LAM, when combined with the strengths GC/MS detection and sample pretreatment, has both clinical sensitivity and clinical specificity. Thus, the methods disclosed herein represent an ultrasensitive detection motif for use in NTM diagnostics, which is also extensible, thereby opening the possibility for the simultaneous detection of multiple markers as a means to further enhance the sensitivity and specificity of the tests.

In addition, low-cost-per-test diagnostics/detection kits for NTM, which incorporate stable regents (e.g., calibration standards, derivatization reagents, pre-made capture substrates, etc.) and materials for urine pretreatment, will be useful in remote or poverty areas.

In the methods disclosed herein, a biological sample, such as a urine sample, is obtained from a patient having a respiratory disease or condition, such as CF, and/or from patients who are susceptible to NTM infection; the presence of LAM is determined by GC/MS analysis on at least a portion of the biological sample. In these methods, a portion of the biological sample is modified to reveal molecular derivatives of LAM that can be specifically and selectively detected by GC/MS. Detection of LAM, or derivatives thereof, from the biological sample indicates an NTM infection in the patient from whom the biological sample was obtained.

The biological sample may be unprocessed and untreated for use within minutes of collection. Alternatively, the sample may be stored and/or shipped to a testing location. For example, the sample may first be frozen shortly after collection from the patient, and then stored and/or shipped for testing by GC/MS at a later date. While the detection methods of this disclosure are sufficiently sensitive such that there is no need to filter or concentrate or culture the sample prior to testing, such filtration and/or concentration of the sample may take place if that is needed or helpful during storage of the sample prior to testing. When using urine as the biological sample, only a small sample of urine from the patient is required for this testing, having the advantageous effect of requiring and producing low volumes of bio waste.

In one aspect, a biological sample, such as urine, is modified prior to GC/MS testing. The samples are first modified by hydrophobic interaction chromatography (HIC) over OCTYL SEPHAROSE® chromatography media. This first modification of the sample separates LAM from the stereoisomeric forms D- and L-arabinose containing neutral glycans that are normally present in urine (from nutrients and food). The OCTYL SEPHAROSE®-purified LAM from urine is then processed for GC/MS detection of derivatives of LAM.

In the methods of this disclosure, two processing methods are useful: 1) derivatization to reveal D-arabinosyl; and 2) alkaline hydrolysis to form a pentafluorobenzyl tuberculostearate derivative. For each of these processing methods (derivatization to D-arabinosyl and alkaline hydrolysis to form pentafluorobenzyl tuberculostearate), the derivative is detected by GC/MS and the amount of LAM in the biological sample is calculated from the amount of the derivative detected. These two processing methods can be used individually (i.e., by processing and detection of D-arabinosyl alone, or pentafluorobenzyl tuberculostearate alone) or concurrently (i.e., by derivatization of separate portions of the OCTYL SEPHAROSE®-purified biological sample).

In these methods, the formation of the D-arabinosyl occurs when the OCTYL SEPHAROSE®-purified biological sample (such as when urine is the biological sample) is subjected to acid hydrolysis to release D-arabinose and synthesize 1-(α/β-O—I-2-octyl)-2,3,5 tri-O-trifluoroactyl-D-arabinofurano/pyranoside. D-¹³C₅-UL-arabinose (200 ng) can be used as an internal standard to compare to the diagnostic four peaks arising due to the formation of α/βanomers of the D-arabinopyranosyl and D-arabinofuranosyl ring conformers during derivatization. The D-arabinosyl is then analyzed by GC/MS using MS/MS. The ions m/z 420.9 (parent ion) to 192.9 (daughter ion), and m/z 425.9 (parent ion) to 197.9 (daughter ion) are monitored, respectively, for D-arabinosyl and the internal standard (D-UL-¹³C₅-arabinose). A comparison between D-arabinosyl and the internal standard (D-UL-¹³C₅-arabinose) yields the D-Arabinose content in the biological sample, which is then used to calculate the LAM equivalent in the biological sample. In a preferred aspect, the biological sample is a urine sample.

In these methods, the formation of the pentafluorobenzyl tuberculostearate occurs when the OCTYL SEPHAROSE®-purified biological sample, such as urine, is subjected to alkaline hydrolysis. D2-palmitic acid can be used as an internal standard. GC/MS analysis of the pentafluorobenzoate ester is carried out using selective ion monitoring program in negative ion chemical ionization mode whereby the characteristic free fatty acyl anion (tuberculostearic acid) at m/z 293.7 is monitored. A comparison between tuberculostearic acid (TBSA) and D2-palmitic acid (internal standard; 20 ng; m/z 257.3) yields the TBSA content in the sample, which is then used to calculate the LAM equivalent in the biological sample. In a preferred aspect, the biological sample is a urine sample.

Detection of LAM (as the D-arabinosyl or TBSA derivatives described above) indicates or diagnoses the presence of a NTM infection in the patient from whom the biological sample was obtained. The detection may indicate the presence of mycobacterial species within the M. avium complex (MAC) or subspecies of M abscessus (MABC).

Patients testing positive for the presence of an NTM infection (i.e., being diagnosed as having an NTM infection) using the methods of this disclosure can be treated or offered treatment for an infection, and/or may be further tested for confirmation of the presence of mycobacterial infection (for example, by testing of a sputum sample from the CF patient), and/or can be monitored for clearance of the mycobacterial infection without treatment.

Patients testing negative for the presence of an NTM infection (i.e., being diagnosed as not having an NTM infection) using the methods of this disclosure may be recommended to have no further testing for the presence of an NTM infection. This recommendation may extend for months (1 month, 2 months, 3 months, 4 months, 5 months, 6 months, 7 months, 8 months, 9 months, 10 months, or 11 months) or even a year. For example, a negative test result may result in a recommendation that the patient from whom the urine sample was obtained not undergo further testing/no testing for a mycobacterial infection for at least 6 months, or for at least 1 year. This decrease in unnecessary testing will save patient money while allowing patients who have difficulty producing a sputum sample by expectoration (such as CF patients taking certain drugs commonly used in the treatment of CF) to avoid the inconvenience of providing sputum samples.

The concentration of LAM and/or a derivative thereof can also be determined by the methods disclosed herein in the biological sample, such as in a urine sample. The method is capable of detecting LAM and/or a derivative thereof in the biological sample at concentrations between about 0.01, 1.0, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, to about 10,000 ng/mL. Higher concentrations of LAM and/or derivatives thereof, can be a possible indicator for a more severe form of NTM disease.

This disclosure also provides kits for the assay of LAM and derivatives thereof in a biological sample from a patient having a respiratory disease or condition and/or from patients who are susceptible to NTM infection. In one aspect, the biological sample is a urine sample. These kits can comprise one or more of hydrophobic separation media including but not limited to OCTYL SEPHAROSE® separation media, acids (for example, trifluoroacetic acid (TFA), bases (for example potassium carbonate, sodium hydroxide, potassium hydroxide), internal standards (for example, D-¹³C₅-UL-arabinose and/or D₂-palmitic acid), assay buffers, wash buffers, sample diluents, and standard diluents. These kits can further comprise detection agents for other known Gram-positive or Gram-negative bacteria (for example staphylococcus, pneumococcus, and Hemophilus influenzae). These kits can further comprise detection agents for other known infectious agents common to patients having a respiratory disease or condition, such as CF patients.

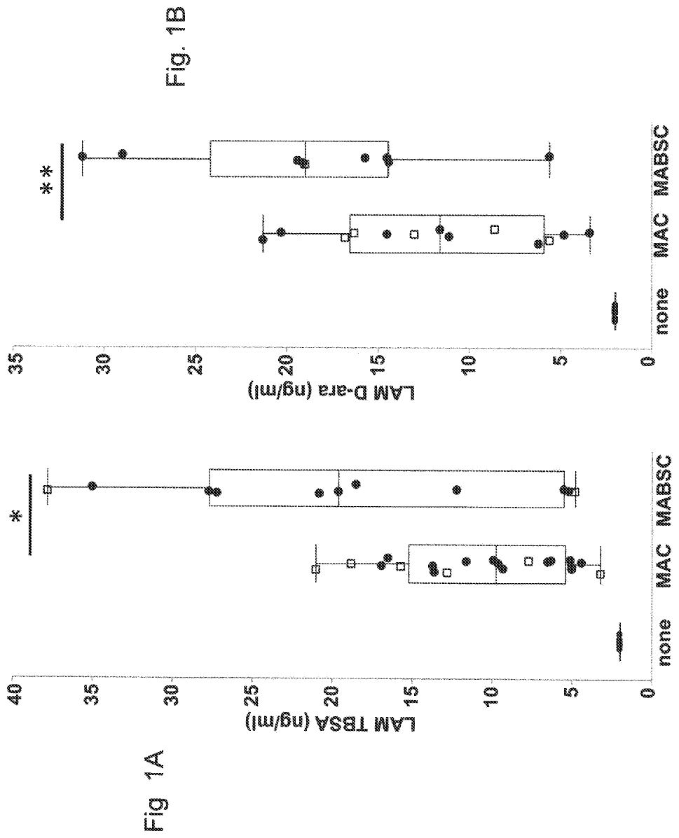

Using a CF cohort well characterized for NTM, the data provided for in the Examples disclosed herein, demonstrate a complete correlation between the presence of LAM in the urine and a history of NTM recovered from sputum samples (Table 1 in the Example section). In these patients, NTM burden ranged from smear and sputum culture positive at the time of urine culture to an individual whose last positive culture was 6.5 years prior. Based on their most recent positive culture, subjects were classified as having either MABSC or MAC, but 9 individuals had a history of more than 1 NTM species, subspecies or complex. The quantity of urinary LAM correlated generally with identification of subspecies of M abscessus or MAC in the most recent sputum sample, with higher quantities associated with M. abscessus (FIGS. 1A and 1B). There was no correlation between urine LAM quantity and traditional markers of disease activity or bacterial burden in this small cohort, including having negative cultures for greater than 1 year, the consensus endpoint for treatment (Floto R A, et al. US Cystic Fibrosis Foundation and European Cystic Fibrosis Society consensus recommendations for the management of non-tuberculous mycobacteria in individuals with cystic fibrosis: executive summary. Thorax 2016; 71:88-90). This lack of correlation between urine LAM and NTM infection status is not unexpected. Although NTM diagnosis and nearly all therapeutic decisions in the CF population depend on sputum culture results, it is well-recognized that sputum cultures are neither sensitive or specific for any aspect of the infection. Evidence of clinical decline attributed to NTM disease can be detected in the year prior to a first positive NTM culture, but the presence of a positive culture does not by itself serve as an indication for treatment. The nearly universal occurrence of bacterial co-pathogens in the CF sputum results in the need for decontamination procedures, which significantly reduce NTM viability and can be the source of false negative results. Likewise, a single sputum sample may not be a representative of infection in all areas of the lung, especially if the NTM is localized within a cavity or in a segment with atelectasis, collapse or extensive mucous plugging.

The singular forms “a”, “an” and “the” include plural referents unless the context clearly dictates otherwise.

Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Where necessary, ranges have been supplied, and those ranges are inclusive of all sub-ranges there between.

Numerical ranges recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

As used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 10%. In all cases, where the term “about” has been used to describe a value, it should be appreciated that this disclosure also supports the exact value.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The described features, structures, or characteristics of the methods, compositions, and kits provided herein may be combined in any suitable manner in one or more embodiments. In the description, numerous specific details are provided, to provide a thorough understanding of embodiments. One skilled in the relevant art will recognize, however, that the embodiments may be practiced without one or more of the specific details, or with other methods, components, materials, and so forth. In other instances, well-known structures, materials, or operations are not shown or described in detail to avoid obscuring aspects of the embodiments.

The contents of all references, patents and published patent applications cited throughout this application, as well as their associated figures are hereby incorporated by reference in their entirety. In case of conflict, the present specification, including its specific definitions, will control.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

The following methods were used to conduct the experiments described in the examples below:

Subjects with CF used to test urine LAM. All subjects were followed by the Colorado CF Center for a minimum of 5 years, with results of at least 5 previous NTM sputum cultures available at time of enrollment. NTM sputum cultures were obtained at the same clinic visit or hospitalization as the urine sample collection. Specimens were digested/decontaminated using a standard NaOH-NALC procedure, and the sediment used to prepare an auramine smear and inoculate various growth medium including: Lowenstein-Jensen Slant, Middlebrook 7H11 Agar/Mitchison 7H11 Selective Agar biplate(s) and MGIT (Mycobacteria Growth Indicator Tube) incubated for up to six weeks. Positive acid fast Bacillus (AFB) culture growth was assessed from solid growth media at 1, 3, and 6 weeks. Mycobacteria Growth Indicator Tube (MGIT) specimens were continuously monitored. Upon growth, detection genomic DNA was isolated for subsequent PCR amplification. A targeted segment of the DNA-directed RNA polymerase subunit beta (rpoβ) gene was performed and a consensus sequence determined, constructed and blasted against a known database to determine molecular identification.

Patients were enrolled either at baseline health or during hospitalization for an acute pulmonary exacerbation. Urine samples were collected in sterile containers and frozen within one hour at −20° C. and then transferred to an −80° C. freezer. Frozen samples were thawed to ambient temperature before assays.

D-Arabinose (D-ara) and Tuberculostearic acid (TBSA) analysis by GC/MS: All urine samples were subjected to hydrophobic interaction chromatography (HIC) over OCTYL SEPHAROSE® (OS)—CL 4B. The 40% and 65% n-propanol in 0.1 M NH₄Oac eluents off the HIC column was processed for GC/MS analysis downstream. For D-arabinose (D-ara) estimation, acid hydrolysis (2MTFA) was carried out to release D-ara and 1-(α/β-O— I-2-octyl)2,3,5 tri-O-trifluoroactyl-D-arabinofurano/pyranoside was synthesized. D-¹³C₅-UL-arabinose (200 ng) was used as an internal standard to compare to the diagnostic four peaks arising due to the formation of α/βanomers of the D-arabinopyranosyl and D-arabinofuranosyl ring conformers during derivatization. The amount of LAM-equivalent was calculated using the following formulation:

LAM Quantitation (D-arabinose): For quantitation, theoretically the ratio of internal standard to sample arabinose can be used for any one of the four isomeric peaks of the ¹³C₅-D-arabinose/¹²C₅-D-arabinose since both stable isotope-labeled internal standard and the unlabeled D-arabinose in the sample will give identical ratios of the α and βanomers of the pyranosyl and furanosyl ring forms for a given sample (the ratio may vary between samples). All four peaks were clean of contaminants as shown by a consistency of the ¹²C-D-arabinose/¹³C-D-arabinose (200 ng used) and therefore all four peaks were used in the calculation. This led to Equation SM-1 to determine the amount of arabinose in the tube:

$${A}_{0} = \frac{1}{4} ( \frac{{p}_{1}}{{I}_{1}} + \frac{{p}_{2}}{{I}_{2}} + \frac{{p}_{3}}{{I}_{3}} + \frac{{p}_{4}}{{I}_{4}} ) \times 200 ng \qquad [ Eqn . S - 1 ]$$

where A₀ is the amount of D-arabinose in the tube in nanograms. (p₁, p₂, p₃, p₄) are the peak areas of the ion from ¹²C₅-D-arabinose and (I₁, I₂, I₃, I₄) are the areas of the ion from the internal standard ¹³C₅-D-arabinose. The amount of LAM per milliliter (L₁, n=volume of urine used in mL) was then calculated by Equation SM-2, after consideration that the mass contribution of D-arabinose to full-length LAM is 60%:

$${L}_{1} = \frac{{A}_{0}}{0.6} / ( n ) ng / mL \qquad [ Eqn . S - 2 ]$$

LAM Quantitation (Tuberculostearic Acid, TBSA). First, the amount of detected TBSA (T₀) is calculated using the following equation SM-3

$$Eqn S - 3 ( Amount of Internal Standard used 20 ng )$$

$${T}_{0} = \frac{Area m / z 297 \text{.3}}{Area m / z 257 \text{.3}} \times 20 ng$$

The approximate molecular weight of full-length LAM is ˜17.3 kDa and the molecular weight of TBSA is 298. Therefore, the mass contribution of TBSA is 298/17300=0.0172 or 1.72%. The amount of estimated LAM equivalent/mL (L₁) is calculated using the following equation, SM-4:

$${L}_{1} = \frac{{T}_{0}}{0.0172 \times n} ng / mL \qquad Eqn . S - 4$$

where n=volume of urine used in mL

The D-ara derivatives were then analyzed by GC/MS using MS/MS. The ions m/z 420.9 (parent ion) to 192.9 (daughter ion), and m/z 425.9 (parent ion) to 197.9 (daughter ion) were monitored respectively for D-ara and D-UL-¹³C₅-arabinose (internal standard, Cambridge Isotope 164 Laboratories Inc.) as reported earlier (De P, et al. 2015. PloS One 10:e0144088).

For TBSA, the OCTYL SEPHAROSE®-purified LAM from urine was subjected to alkaline hydrolysis and subsequently the corresponding pentafluorobenzyl tuberculostearate derivative was made. D2-palmitic acid was used as the internal standard. The GC/MS analysis of the pentafluorobenzoate ester was carried out using selective ion monitoring program in negative ion chemical ionization mode whereby the characteristic free fatty acyl anion at m/z 293.7 was monitored. A comparison between TBSA and D2-palmitic acid (internal standard; 20 ng; m/z 257.3) yields the TBSA content in the sample, which was then used to calculate the LAM equivalent. GC/MS analyses were carried out using a Thermo GC-TSQ™8000 Evo Triple Quad GC mass spectrometer. Chromatograms with respective peaks were integrated manually (i.e., peak areas were defined manually and integrated areas were generated by the computer software) for the estimation of total D-ara and TBSA content. The instrument was set to collect data for m/z 257.3 in the range of 5 to 19 minutes for internal standard (ISTD) and m/z 297.3 after 19 minutes for TBSA which elutes around 20 minutes, to collect sufficient data points for low level mass detection.

Capture ELISA: A polystyrene microplate was coated with 100 μl of a capture antibody at 10 ug/mL concentration in PBS and incubated at 4° C. overnight. Urine control samples were spiked with known amount of LAM at different concentrations and incubated at 4° C. overnight to allow for the complexation of LAM and protein/s. Clinical as well as control samples were pretreated with Proteinase K and the supernatant used for ELISA. After overnight incubation, the antibody coated plates and the LAM samples were brought to room temperature (RT) and the plates were blocked for 1 hr at room temperature. The plates were washed with the wash buffer (200 ul×10) and the control and the clinical samples were added to the appropriate wells (100 ul) and incubated. Following a second wash, the plates were incubated with the biotinylated detection antibody at a final concentration of 250 ng/mL in wash buffer. Biotinylation of the antibody was carried out using EZ-LINK™ Sulfo NHS-LC Biotin (THERMOFISHER SCIENTIFIC™) following the kit protocol and the labelled antibody was desalted on ZEBA™ spin desalting columns, 7K MWCO (THERMOFISHER SCIENTIFIC™) as per the kit protocol. Following a third wash, 100 ul of 1:200 dilution of Streptavidin-Horseradish Peroxidase (HRP) (R & D Systems) was added to the plates and incubated for 25 min. After the final wash, 100 ul Ultra TMB-ELISA chromogenic substrate (THERMOFISHER SCIENTIFIC™) was added to the plates and incubated for 30 min. Reaction was stopped by addition of Sulphuric acid (FISHER SCIENTIFIC™) and the absorbance was read at 450 nm. All the controls were run in triplicates and reported as the mean±standard deviation. The samples were run in duplicates and plotted against the standard curve generated by spiking the urine from a healthy volunteer with known amounts of LAM and serially diluted. Limits of blank (LoB) and limits of detection (LoD) were generated from the standard curve using CLSI standard.

LAM and mAbs used for standardization of immunoassay: The LAM used in this study was isolated and purified from M tuberculosis CDCl551 and H37Rv, M avium 2285 rough in vitro cultures. The LepLAM was purified from M leprae whole cells isolated from infected armadillo spleen and liver. LAM from all the above-mentioned sources was prepared as described in Mycobacterial Protocols, Lind E D (Shi L, et al. 2009. Lipoglycans of Mycobacterium tuberculosis: isolation, purification, and characterization, p 23-45. In Brown TpaAC (ed), Mycobacteria Protocols, second edition ed, vol 465. Humana Press, Barts and The London, London, UK.). Characterization and purification of all monoclonal antibodies (mAbs) was done as described in Gaylord H, et al. Most Mycobacterium leprae carbohydrate-reactive monoclonal antibodies are directed to lipoarabinomannan. InfectImmun 1987; 55:2860-3; and Rivoire B, et al., Aspinall G O, Brennan P J. Generation of monoclonal antibodies to the specific sugar epitopes of Mycobacterium avium complex serovars. InfectImmun 1989; 57:3147-58.

### Example 1

### NTM Characteristics of CF Population

Forty-five urine samples were analyzed from 44 patients with well-documented NTM culture status. Patients were representative of the CF population in general, ranging in age from 11 to 63 years (mean=24.6 years), with 16 men and 28 women. Thirty-one subjects had a history of a positive NTM sputum culture (Table 1) (11 MABSC, 20 MAC) ranging from 0 to 2385 days prior to urine collection. Sixteen subjects had a positive NTM sputum culture at the same clinic visit or hospitalization as their urine collection, and 1 was smear positive. Among the 31 patients with a history of positive NTM cultures, 7 had been treated and were culture negative for more than a one year and were classified as having “cleared” their sputum, while the remaining 24 were culture positive within the last year or had never received NTM treatment. Fourteen subjects had a history of infection with more than one NTM species or complex (Table 1). All patients were co-infected with at least one other typical CF pathogen, including Pseudomonas aeruginosa, Staphylococcus aureus, or Aspergillus species.

TABLE 1
GC/MS BASED QUANTIFICATION OF URINE LAM MARKERS TBSA AND D-ARA

| | Culture | Days since | Current or most | Current or most | ID of other NTM | LAM | LAM |
|---|---|---|---|---|---|---|---|
| Subject | Status¹ | last pos. Cx | recent NTM ssp | recent complex | species² | (TBSA)³ | (D-Ara)⁴ |
| BP-001 | Never pos. | N/A⁵ | N/A | none | N/A | ND⁶ | ND |
| BP-002 | Never pos. | N/A | N/A | none | N/A | ND | ND |
| BP-011 | Never pos. | N/A | N/A | none | N/A | ND | ND |
| BP-202 | Never pos. | N/A | N/A | none | N/A | ND | ND |
| BP-204 | Never pos. | N/A | N/A | none | N/A | ND | ND |
| BP-209 | Never pos. | N/A | N/A | none | N/A | ND | ND |
| BP-210 | Never pos. | N/A | N/A | none | N/A | ND | ND |
| BP-308 | Never pos. | N/A | N/A | none | N/A | ND | ND |
| BP-309 | Never pos. | N/A | N/A | none | N/A | ND | ND |
| BP-310 | Never pos. | N/A | N/A | none | N/A | ND | ND |
| BP 501 | Never pos. | N/A | N/A | none | N/A | ND | ND |
| BP502 | Never pos. | N/A | N/A | None | N/A | ND | ND |
| BP 503 | Never pos. | N/A | N/A | None | N/A | ND | ND |
| BP 505 | Never pos. | N/A | N/A | none | N/A | ND | ND |
| BP-208 | Cleared | 2385 | MAC (unspecif) | MAC | none | 21.0 | 16.3 |
| BP-008 | Cleared | 1095 | M. abscessus | MABSC | M. avium | 37.8 | 19 |
| TCT-008 | Cleared | 813 | M. abscessus | MABSC | None | 4.8 | Na⁷ |
| BP-301 | Cleared | 634 | M. avium | MAC | M. abscessus, | 3.2 | 5.6 |
| | | | | | M. gordonae | | |
| BP-311 | Cleared | 601 | M intracellulare | MAC | none | 18.8 | 16.8 |
| BP-403 | Negative | 469 | M. chimaera | MAC | None | 7.8 | 11.0 |
| BP-305 | Cleared | 447 | M. avium | MAC | M. avium | 12.8 | 8.6 |
| BP-004 | Cleared | 391 | M. intracellulare | MAC | M. abscessus | 15.7 | 13 |
| TCT-003 | Negative | 236 | M. avium | MAC | M. triplex | 4.4 | na |
| | | | | | M. montefiorence | | |
| BP-009 | Positive | 91 | M. intracellulare/ | MAC | M. massiliense | 5 | 4.8 |
| | | | yongonense | | | | |
| BP-304 | Positive | 88 | M. avium | MAC | M. abscessus, | 9.3 | 11.1 |
| | | | | | M gordonae | | |
| BP-406 | Negative | 88 | M. chimaera | MAC | M. avium | 15.4 | 12.6 |
| BP-404 | Negative | 78 | M. avium | MAC | None | 10.3 | 11.3 |
| BP-003 | Positive | 52 | M. abscessus | MABSC | none | 5.2 | 5.6 |
| BP-201 | Positive | 23 | M. massiliense | MABSC | none | 35 | 29 |
| BP-303 | Positive | 8 | M. massiliense | MABSC | none | 12.2 | 19.1 |
| BP-306 | Positive | 6 | M intracellulare | MAC | M avium, | 16.5 | 21.3 |
| | | | | | M. yongonense, | | |
| | | | | | M. abscessus | | |
| BP-203 | Positive | 4 | M. abscessus | MABSC | none | 18.5 | 14.4 |
| BP-011 | Postive⁸ | 4 | M. avium | MAC | None | 5.1 | na |
| BP-405 | Positive | 2 | M. abscessus | MABSC | None | 8.2 | 9.2 |
| BP-307 | Positive | 1 | M. avium | MAC | M. massiliense, | 16.9 | 14.5 |
| | | | | | M. chimaera | | |
| BP-401 | Positive | 1 | M. intracellulare | MAC | None | 8.0 | 8.6 |
| BP-005 | Positive | 0 | M. intracellulare | MAC | none | 9.6 | 6.2 |
| BP-006 | Positive | 0 | M. abscessus | MABSC | M. avium | 27.2 | 19.4 |
| BP-007 | Positive | 0 | M. avium | MAC | M. intracellulare | 5 | 3.4 |
| BP-010 | Positive⁹ | 0 | M. abscessus | MABSC | none | 27.7 | 31.2 |
| BP-205 | Positive | 0 | M. abscessus | MABSC | none | 19.6 | 15.7 |
| BP-206 | Positive | 0 | M. abscessus | MABSC | none | 20.8 | 14.5 |
| BP-207 | Positive | 0 | M. intracellulare/ | MAC | none | 13.7 | 20.3 |
| | | | yongonense | | | | |
| BP-302 | Positive | 0 | M. avium | MAC | M. massiliense | 9.9 | 11.6 |
| BP-402 | Positive | 0 | M. chimaera | MAC | M. abscessus | 13.6 | na |

¹Culture Status: Never pos. represents a minimum of 5 NTM cultures over the 5 years previous to collection, as well as no known history of a previous NTM culture; Cleared represents NTM sputum cultures negative for >1 year, Positive represents NTM sputum cultures positive within the last year. Negative represents sputum cultures that were previously positive but now negative, either with less than a year of treatment of spontaneously.
²ID of other NTM species: Identification of NTM in subjects with a lifetime history of >1 species or subspecies prior to most recent culture.
³LAM (TBSA): Urine LAM TBSA quantity as ng/ml.
⁴LAM (D-ara): Urine LAM D-ara quantity as ng/ml.
⁵N/A: Not applicable
⁶ND: Not Detected.
⁷na: Not available due to insufficient quantity of sample.
⁸Subject tested twice, first classified as never positive, and then retest at the time of positive sputum cultures for MAC.
⁹AFB smear positive on date of urine collection

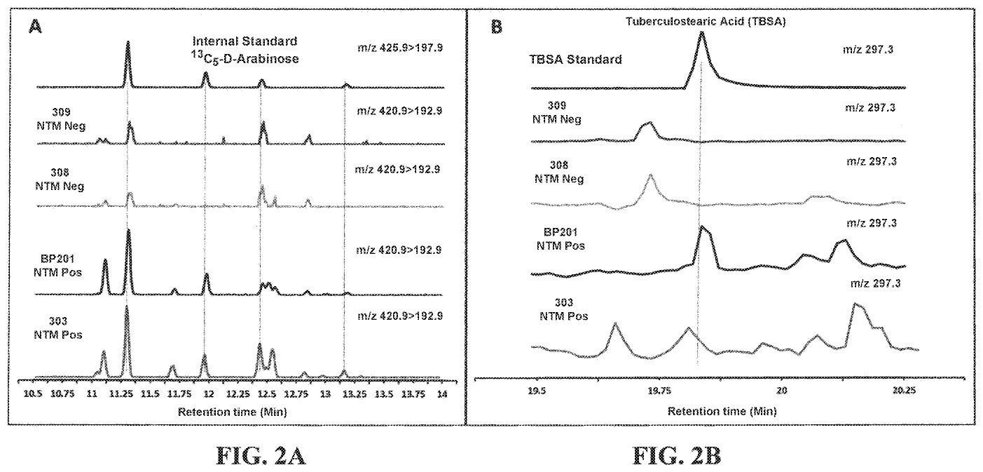

Correlation of urine LAM with NTM phenotype and species: All subjects with a known previous positive sputum culture for NTM had detectable urine LAM through GC/MS analysis of either D-ara or TBSA (Table 1). Among patients whose current or most recent infection was a subspecies of M abscessus, the quantity of both TBSA and D-ara were significantly greater than those infected with MAC (p=0.0073 and p=0.03 for TBSA and D-ara, respectively) (FIGS. 1A and 1B). Interestingly, there was no clear correlation between quantity of detected LAM and culture status, as subjects who had positive cultures within a year of urine collection had a similar range of LAM detected as subjects who had cleared their sputum culture for greater than 1 year (FIGS. 1A and 1B). Representative GC/MS chromatograms showing the absence and/or presence of urinary LAM related to NTM-negativity and/or positivity are shown in FIGS. 2A and 2B.

### Example 2

### Predictive Value of Negative LAM Result

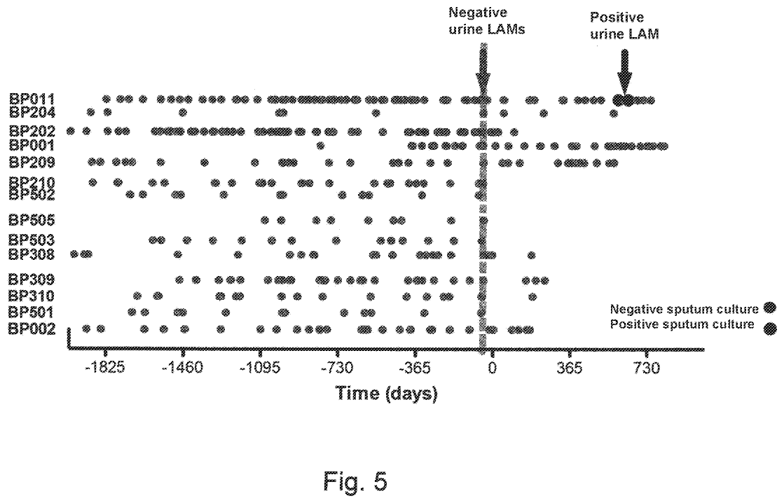

In order to better understand the predictive value of a negative urine LAM result, the NTM sputum culture history was examined in detail for the fourteen subjects classified as never having a positive NTM sputum culture. In total, these subjects had 407 negative NTM sputum cultures over a mean interval of 2.75 years (range 0 to 9.9 years) (FIG. 5). As enrollment in this study occurred over a two-year period, follow-up culture results were also available for some subjects after urine LAM analysis. Subjects had follow-up sputum cultures, with a mean of 10 (range 1 to 27) cultures per subject and a median follow-up time of 12.75 months (FIG. 5). Over the course of trial enrollment one of the subjects (BP011) initially classified as negative subsequently developed NTM disease with 3 positive cultures forts. avium occurring 657 days after the initial urine LAM testing. Repeat urine LAM testing on this subject determined that her urine LAM assay turned positive at the time of her positive NTM cultures, supporting the ability of this assay to detect a conversion from sputum culture negative to positive. The subject met criteria for NTM disease and was started on antibiotic treatment with conversion of her cultures to negative (FIG. 5).

### Example 3

### Tuberculosis (TB) ELISA on NTM Clinical Samples

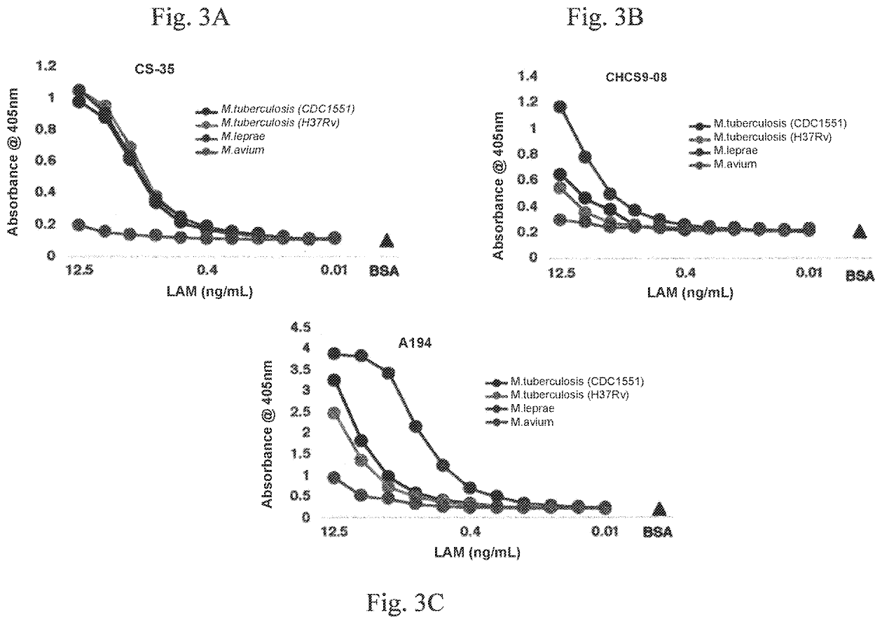

Urine samples described in Table 1 were also tested via a recently described capture ELISA that was developed for the purpose of TB diagnosis (Amin et al. 2018. Tuberculosis (Edinb) 111:178-87). Three antibodies (CHCS9-08, CS-35 and A194-01) were selected that worked well with the M. tuberculosis clinical samples. Prior to running the CF clinical samples, the three antibodies were tested individually against serial dilution of LAM from M tuberculosis, M leprae and M avium. M avium LAM shares many common structural features with TBLAM (Khoo K-H, et al. Variation in mannose-capped terminal arabinan motifs of lipoarabinomannans from clinical isolates of Mycobacterium tuberculosis and Mycobacterium avium complex. J Biol Chem 2001; 276:3863-71.), and the species are more closely related to each other than with M abscessus. The antibodies were selected based on the following evaluation (see FIG. 3). Each antibody had significantly lower affinity towards NTMLAM compared to LepLAM, or TBLAM, despite having selected antibodies from a collection that have broad epitope binding affinity.

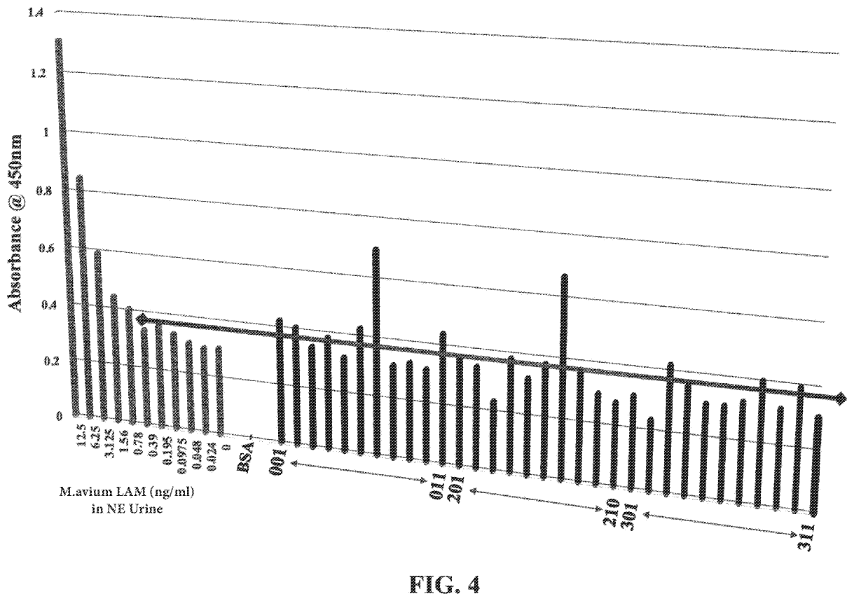

All the forty-five NTM urine samples that had previously been analyzed by GC/MS were tested in an ELISA format following pretreatment with Pro K. LAM was detected in only two of the samples from NTM positive subjects, and none of the NTM negative subjects by immunoassay irrespective of any combination of antibodies used (FIG. 4).

The various features and processes described above may be used independently of one another or may be combined in various ways. All possible combinations and sub-combinations are intended to fall within the scope of this disclosure. The example systems and components described herein may be configured differently than described. For example, elements may be added to, removed from, or rearranged compared to the disclosed example embodiments.

While certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

1. A method for diagnosing nontuberculosis mycobacterial (NTM) infection in a patient, the method comprising:
a. obtaining a biological sample from the patient;
b. detecting lipoarabinomannan (LAM) or a derivative thereof in the biological sample from the patient, wherein the LAM or a derivative thereof is detected by gas-chromatography/mass spectrometry (GC/MS);
wherein detection of the LAM or a derivative thereof in the biological sample from the patient diagnoses the patient as having NTM infection,
wherein the patient diagnosed as having NTM infection is:
1. treated for the NTM infection,
2. further tested for NTM infection in a sputum sample from the patient, and/or
3. monitored for clearance of the NTM infection;
or wherein non-detection of the LAM or a derivative thereof in the biological sample from the patient diagnoses the patient as not having NTM infection, and wherein the patient is not tested again for the presence of NTM infection for at least 6 months or for at least 1 year.

2. The method of claim 1, wherein the patient has a respiratory disease or condition selected from the group consisting of cystic fibrosis (CF), asthma, chronic obstructive pulmonary disease (COPD), bronchiectasis, silicosis, sarcoidosis, pneumoconiosis, and interstitial lung disease.

3. The method of claim 2, wherein the respiratory disease or condition is cystic fibrosis.

4. The method of claim 1, wherein the biological sample is a urine sample.

5. The method of claim 1, further comprising purifying the biological sample by hydrophobic interaction chromatography (HIC).

6. The method of claim 5, wherein the HIC comprises contacting the biological sample with OCTYL SEPHAROSE® chromatography media.

7. The method of claim 1, wherein the derivative of LAM is D-arabinosyl.

8. The method of claim 7, further comprising forming D-arabinosyl in the biological sample by contacting the sample with an acid.

9. The method of claim 8, wherein the acid is trifluoracetic acid.

10. (canceled)

11. The method of claim 1, further comprising use of D-UL-¹³C₅-arabinose internal standard in the GC/MS or D₂-palmitic acid internal standard in the GC/MS.

12. The method of claim 1, wherein the derivative of LAM is tuberculostearic acid (TBSA).

13. The method of claim 12, further comprising forming TBSA in the biological sample by contacting the sample with a base.

14. The method of claim 13, wherein the biological sample undergoes alkaline hydrolysis.

15.-16. (canceled)

17. The method of claim 1, further comprising determining the LAM and/or a derivative thereof concentration in the biological sample.

18. The method of claim 17, wherein the method detects the concentration of the LAM and/or a derivative thereof in the biological sample at concentrations between about 0.01 to about 10,000 ng/mL.

19.-23. (canceled)

24. A method for detecting LAM in a biological sample, the method comprising:
a. obtaining a biological sample; and
b. detecting lipoarabinomannan (LAM) or a derivative thereof in the biological sample, wherein the LAM or a derivative thereof is detected by gas-chromatography/mass spectrometry (GC/MS).

25. The method of claim 24, wherein the biological sample is obtained from a patient having a respiratory disease or condition selected from the group consisting of CF, asthma, COPD, bronchiectasis, silicosis, sarcoidosis, pneumoconiosis, and interstitial lung disease.

26.-27. (canceled)

28. The method of claim 24, further comprising purifying the biological sample by hydrophobic interaction chromatography (HIC).

29. (canceled)

30. The method of claim 24, wherein the derivative of LAM is D-arabinosyl or tuberculostearic acid (TBSA).

31.-41. (canceled)

42. A kit for detecting LAM or a derivative thereof in a biological sample using the method of claim 24, the kit comprising one or more of OCTYL SEPHAROSE® separation media, acids, bases, internal standards, assay buffers, wash buffers, sample diluents, and standard diluents.

43.-47. (canceled)
